(19) United States
(12) Patent Application Publication
(10) Pub. No.: US 2013/0116750 A1
(43) Pub. Date: May 9, 2013

(54) METHOD FOR SAFEGUARDING IMPLANTED MEDICAL DEVICES IN A DIAGNOSTIC DEVICE EMITTING ELECTROMAGNETIC RADIATION

(71) Applicants: Markus Schmidt, Nuernberg (DE); Jörg-Thomas Steiner, Erlangen (DE); Christoph Zindel, Knoxville, TN (US)

(72) Inventors: Markus Schmidt, Nuernberg (DE); Jörg-Thomas Steiner, Erlangen (DE); Christoph Zindel, Knoxville, TN (US)

(21) Appl. No.: 13/671,750

(22) Filed: Nov. 8, 2012

(30) Foreign Application Priority Data
Nov. 9, 2011 (DE) 102011085975.6

(51) Int. Cl. A61N 1/02

(52) U.S. Cl. CPC A61N 1/025
USPC 607/59; 607/62

(57) ABSTRACT

A method for safeguarding an implanted medical device from the electromagnetic radiation from a diagnostic device is provided. The method includes detecting the implanted medical device by a bidirectional communication with the diagnostic device, determining a device type of the medical device with the diagnostic device, checking whether the device type of the medical device may be operated without errors under the electromagnetic radiation of the diagnostic device and initiating a protective measure if the device type of the medical device cannot be operated without errors under the electromagnetic radiation.

### CROSS REFERENCE TO RELATED APPLICATIONS

This application claims priority of European Patent Office application No. 10 2011 085 975.6 DE filed Nov. 9, 2011. All of the applications are incorporated by reference herein in their entirety.

### FIELD OF INVENTION

A method for safeguarding an implanted medical device for electrotherapy from electromagnetic radiation from a diagnostic device, a control device for carrying out the method, an apparatus with the control device and a network with the apparatus is provided.

### BACKGROUND OF INVENTION

Examination of a patient with an implanted medical device, such as a heart pacemaker, in a nuclear magnetic resonance tomography apparatus, abbreviated to MRI apparatus, is known from DE 60 2004 009 704 T2. The MRI apparatus thus serves as a diagnostic device. The MRI apparatus comprises a control device which can use the medical device to expand the data spectrum for a diagnosis. Data is exchanged wirelessly between the implanted medical device and the control device. In this process the wireless exchange of data between the implanted medical device and the control device is halted during the irradiation burst of the MRI apparatus, so that malfunctions of the implanted medical device can be avoided during an irradiation burst of the MRI apparatus.

Also known from DE 10 2006 043 A1 is a safeguarding device to establish a possible risk to a person from a technical device, wherein a RFID transponder captures personal data about the implanted medical device and the technical device is investigated for possible risk. If a risk is identified, a warning signal is output.

### SUMMARY OF INVENTION

An object is to arrange for an implanted medical device to be used more safely in a diagnostic device emitting electromagnetic radiation.

This object is achieved by the features of the independent claims. Preferred embodiments are the subject matter of the dependent claims.

An underlying idea is that the electromagnetic radiation of the diagnostic device cannot just disturb the implanted medical device in its functionality, it can damage the implanted medical device itself or damage the tissue of the patient by way of the implanted medical device, since induction currents created by the electromagnetic radiation heat up the implanted medical device. This not only represents a significant health risk for the patient but can also lead to financial damage, since the implanted medical device has to be repaired or even replaced.

A further idea is that the effects of the electromagnetic radiation are dependent on the device type of the implanted medical device. A device type in this case is to be understood as the concrete technical embodiment or configuration of a technical device, which can for example be dependent on the manufacturer and/or the version of the technical device. Thus for example one device type of the implanted medical device with many closed circuits promotes the propagation of the induction currents, while another device type tends to avoid the propagation of the induction currents.

Although it can be found out whether or not the electromagnetic radiation of a diagnostic device is harmless for a specific device type of an implanted medical device, this involves taking account of a large amount of information about the implanted medical device. Although this information about the operating instructions of the implanted medical device can be looked up, it can prove difficult however to map the information of the operating instructions to the general conditions provided by the diagnostic device. Ultimately the responsibility lies with the patient themselves to collect and make available all information about their medical device, so that it is possible to evaluate whether their implanted medical device can be operated in the diagnostic device. The problem gets worse if the patient incorrectly identifies their implanted medical device and thus provides incorrect information in the determination of whether or not the electromagnetic radiation of the diagnostic device is damaging for the implanted medical device. In addition the information provided by the patient could be interpreted incorrectly by the doctor.

By contrast, an embodiment proposes that the device type of the implanted medical device be determined by bidirectional communication with the diagnostic device. This makes it possible to automate the decision as to whether the implanted medical device can be operated without problems in the diagnostic device or not, since the necessary information can be captured directly by the computer instead of by the patient or by intermediate steps by human beings. In this way sources of errors are avoided and necessary protection measures for safeguarding the implanted medical device from electromagnetic radiation of the diagnostic device can be initiated in a more appropriate manner

A method is thus specified for safeguarding an implanted medical device against electromagnetic radiation from the diagnostic device. The specified method comprises the steps of detection of the implanted medical device by bidirectional communication with the diagnostic device, determining a device type of the medical device with the diagnostic device, checking whether the device type of the medical device can be operated without errors under the electromagnetic radiation of the diagnostic device and initiating a protective measure if the device type of the medical device cannot be operated without errors under the electromagnetic radiation.

The device type of the implanted medical device can be determined in any given technical manner. Thus the device type of the implanted medical device can be determined for example based on an imaging diagnostic method which is harmless for the implanted medical device. As an alternative or in addition, databases can be set up for the implanted medical device which link the patient with the device type of the implanted medical device. Further options for technically determining the device type of the implanted medical device are specified in the subclaims.

The implanted medical device can be any technical supplementary device which is able to be implanted in the body of a patient for medical support. Thus the device can supply active therapeutic help, such as defibrillators or stimulators for example, which are known for example in the form of heart, brain or muscle stimulators. Further implanted dispensers or pumps for medicines can provide active therapeutic help. As an alternative or in addition, the implanted medical device can provide passive therapeutic help, by recording data about the patient or monitoring the patient for example. These devices can be heart beat recorders or insulin monitoring devices for example.

The diagnostic device can be any diagnostic device emitting electromagnetic radiation, such as a magnetic resonance tomograph for example, for presenting structure and function of the tissue and organs in the body or a magnetic resonance spectroscope for identification and quantification of different chemical substances in living tissue as a result of their chemical displacement.

The protective measure can be any given measure which helps to protect the implanted medical device from damage by electromagnetic radiation of the diagnostic device. The protective measure can thus comprise an automatic block that prevents the diagnostic device being put into operation if an unsuitable implanted medical device is determined. Further protective measures are the subject matter of the subclaims. In particular such a protective measure is a protective measure directed towards the implanted medical device.

The specified method enables patients with an implanted medical device to be protected during examination in a diagnostic device emitting electromagnetic radiation against damage to their tissue or to the implanted medical device, which not only avoids health damage but also economic damage. In addition the specified method represents a lightening of the load on medical personnel, since the automatic verification of the implanted medical device renders manual checks of the specifications for the implanted medical device obsolete.

Bidirectional communication allows not only protective measures to be initiated on the implanted medical device side, but also allows desired device-specific data to be selectively obtained on request.

In a corresponding development the protective measure comprises a reconfiguration of the medical device such that the medical device can be operated without errors under the electromagnetic radiation of the diagnostic device. The bidirectional communication is included for this device-specific protective measure.

In an additional development the diagnostic device is put into operation after the reconfiguration of the medical device. Subsequently the medical device is returned to its original program. In this way the diagnostic process can be carried out in a largely automated manner without the medical personnel needing to intervene.

In another development the device type of the medical device is able to be determined on the basis of an identifier stored in the medical device. The identifier can be any feature uniquely identifying the device type. Thus for example the identifier can be a specific feature which is only stored for the purposes of identification in the medical device. As an alternative or in addition, the features already present can be included as the feature uniquely identifying the device, such as the network address of the implanted medical device or its serial number for example. By using the identifier it is possible to identify the implanted medical device immediately and uniquely.

In an additional development the specified method comprises the step of interrogating the identifier of the medical device and performing the determination of the device type of the medical device based on the interrogated identifier. By using the identifier to determine the device, information is available which can be used to derive further information about the implanted medical device on the basis of a link. The link enables safety-relevant information to be derived about the implanted medical device.

This can for example be the maximum magnetic field strength able to be used or the maximum permitted edge slope steepness of the magnetic field.

In another or an additional development an entry is stored in a database as to whether the device type of the medical device can be operated without errors under electromagnetic radiation. The database can be stored internally in a controller performing the method or externally. The database enables a central information medium to be created from which the patient or a doctor can obtain background information about an implanted medical device in an easy-to-understand manner. The manufacturers of the implantable medical devices and the manufacturers of the diagnostic devices can work together in equal part on the creation of the database, in order to provide the necessary information for safeguarding an implanted medical device against a diagnostic device emitting electromagnetic radiation.

In an additional development the specified method comprises the steps of retrieving the entry from the database on the diagnostic device and checking whether the device type of the medical device can be operated without errors under the electromagnetic radiation of the diagnostic device based on the retrieved entry. The use of the database means that all necessary information for checking whether the device type of the medical device can be operated without errors under the electromagnetic radiation is available through one single search enquiry. Further technical preparation, processing and/or evaluation steps are not necessary.

In a further development the specified method comprises the step of retrieving a number of entries from the database before the step of detecting the implanted medical device. This means that different entries are collected for different device types of the implanted medical device before a patient with an implanted medical device comes into the vicinity of the diagnostic device. In this way the execution of the specified method is rendered independent of the accessibility of the database with the entries for the relevant device type of the implanted medical device.

In an alternative or additional development the method comprises the step of determining an access address in a network based on the identifier and interrogating the entry from the access address. The access address can for example be the network address of the manufacturer of the medical device, who provides the necessary information on their own server as to whether and how the implanted medical device can be operated during diagnosis with the diagnostic device. In this way it is ensured that the most up-to-date information is always available for initiating the protective measures. Furthermore medical devices for which the manufacturers are not involved in the creation of a database can also be taken into account.

In a further development the entry further specifies whether the device type of the medical device can be operated without errors under a specific configuration of the diagnostic device and/or under a specific configuration of the medical device. In this way specific preparation measures at the diagnostic device for safeguarding the implanted medical device can likewise be seen from the database from the access address, such as the server of the manufacturer. The specific configurations can for example involve features that have to be activated in advance on the implanted medical device for carrying out the diagnosis with the diagnostic device.

In another development the protective measure comprises output of a warning as to whether the device type of the medical device can be operated without errors under the electromagnetic radiation. In this way the medical operator operating the diagnostic device can decide themselves about the necessary safeguarding steps.

In an alternative or additional development the protective measure comprises a reconfiguration of the diagnostic device such that the device type of the medical device can be operated without errors under the electromagnetic radiation of the diagnostic device. In this way possible configuration measures to prepare for diagnosis with the diagnostic device without further human interaction can run with corresponding associated error sources. For example the reconfiguration can preferably comprise an enabling request which the medical personnel must confirm so that a reconfiguration can be carried out. In this way the medical personnel have the option of intervening in the process at any time and if necessary making changes to the configuration proposed by the specified method.

A controller is also specified for safeguarding an implanted medical device for electrotherapy against electromagnetic radiation from the diagnostic device, wherein the control device includes a network interface for bidirectional data exchange with the medical device and is set up to execute a specified method.

In particular the data from the medical device is retrieved via the network interface, from which the device type of the medical device is obtained. The network interface can be any given bidirectional data interface, such as Bluetooth or WLAN (wireless local area network).

In an additional development the control device includes a network interface for receiving the entries of the database from a memory in which the database is stored. The network interface can be the same network interface as the network interface for receiving data from the medical device, from which the device type of the medical device is obtained. As an alternative it can also differ from the latter.

An apparatus is also specified for diagnostic examination of the patient which comprises a specified control device and the diagnostic device.

A network is also specified that comprises a specified apparatus and a database with entries, from which it can be seen whether the device type can be operated without errors under the electromagnetic radiation.

### BRIEF DESCRIPTION OF THE DRAWINGS

The properties, features and advantages described above, as well as the manner in which these are achieved will be explained more clearly and in a more readily understandable manner in conjunction with the description of the exemplary embodiments given below, which is explained in greater detail in connection with the drawings, in which:

FIG. 1 shows a network with a control device for executing the specified method.

### DETAILED DESCRIPTION OF INVENTION

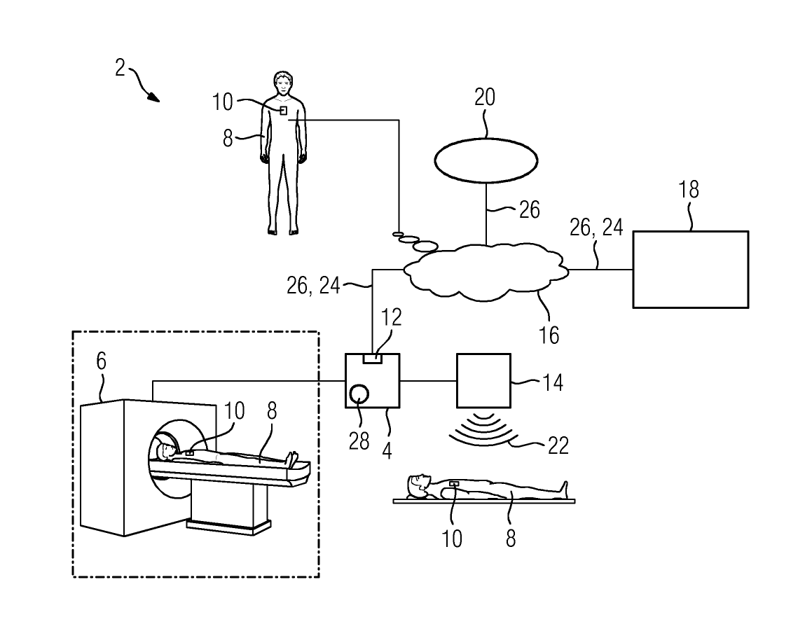

Reference is made to FIG. 1, which shows a network 2 with a control device 4 for executing the specified method.

The control device 4 is intended to control a medical diagnostic device 6, which in the present example is a magnetic resonance tomograph 6, abbreviated to MRT 6. The MRT 6 is primarily used in medical diagnostics for presenting the structure and function of tissue and organs in the body of a patient 8.

Implanted in the patient 8 for therapeutic purposes is a medical device 10. In the present embodiment the medical device 10 is a heart pacemaker 10, abbreviated to HSM 10. The HSM 10 is used to treat heartbeats that are too slow (Bradycardia). To this end the HSM 10 regularly stimulates the heart muscle of the patient 8 with the aid of electrical impulses and in this way excites the heart muscles into contraction.

The control device 4 has two network interfaces 12, 14. A first network interface 12 serves as a connection to the Internet 16. The connection to the Internet 16 can be protected for example by a firewall in a way not shown in the figure. A second network interface 14 in the form of a Bluetooth interface 14 can serve as a wireless communication interface to the HSM 10.

Access to a control unit for device management 18 of the manufacturer of the HSM 10 and a database 20 is possible over the Internet 16.

The control unit for device management 18 stores information provided by the manufacturer in relation to the HSM 10. This information comprises on the one hand whether the HSM 10 may be operated at all in the MRT 6. In addition the information provided can include safety-relevant specifications about the HSM 10 itself, meaning information about the magnetic field strength or the maximum magnetic slope steepness with which the MRT 6 may be operated if the HSM 10 is located therein or which tomography sequences are allowed at all for the HSM 10 in the MRT 6. In addition the information can also comprise safety-relevant settings which are necessary at the MRT 6 in order to operate the HSM 10 in the MRT 6.

Parts of the information from the control unit for device management 18 or all information from the control unit for device management 18 for all manufacturers can be stored in the database 20, so that the patient 8 can look up for themselves, based on consulting the Internet 16, whether or not their HSM 10 is compatible with the MRT 6.

A bidirectional network connection 22 exists between the second network interface 14 and the HSM 10. The control device 4 can receive the medical device identification and if necessary further data from the HSM 10 via the bidirectional network connection 22. The medical device identification can for example be the unique device identification (UDI) used in the United States, which will not be discussed in any greater detail here for the sake of brevity. Furthermore the control device 4 can send program instructions to the HSM 10 in the other direction to prepare it for use in the MRT 6 and to reprogram it accordingly.

There is a unidirectional network connection 24 between the first network interface 12 and the control unit for device management 18. Via this network connection 24 the control device 4 can receive the aforementioned safety-relevant information for the HSM 10 from the control unit for the device management 18. This information is especially preferably provided in standardized form with a predefined data format.

There is also a unidirectional connection between the control unit for the device management 18 and/or the first network interface 12 to the database 20, via which the control device 4 and/or the manufacturer of the HSM 10 can store the safety-relevant information described above about the HSM 10 partly or completely, so that ultimately the information source covering all manufacturers described above for different device types of the HSM 10 is produced.

When the network 2 is used, the manufacturer of the HSM 10 initially creates the safety-relevant information for the HSM 10 and stores this on the control unit for device management 18. In concrete terms this safety-relevant information can be divided into different safety levels for example, in order to demonstrate at a very fundamental level whether the safety-relevant restrictions of the HSM 10 apply to specific MRTs 6 or generally to all MRTs 6. A high safety level of safety-relevant information can show in such cases that the information relates to all MRTs 6. Thus it can for example be shown with such information that the HSM only operates without any safety concerns with a magnetic field of 1.5T. A medium safety level of safety-relevant information can indicate in such cases that the information only relates to specific MRTs 6 which for example are proprietary and/or depend on the version of the driver software of the MRT 6. More detailed descriptions in the information enable it to be specified in very concrete terms which safety-relevant settings are necessary at the MRT 6, in order to meet all the technical safety aspects in respect of the HSM 10 while it is being used in the MRT 6. This safety-relevant information can be sent via the unidirectional network connection 24 from the control unit for device management 18 to the control device 4, wherein the control device 4 stores the received safety-relevant information.

If the patient 8 enters a room with the MRT 6, in which the second network interface 14 is also installed, this can detect the HSM 10 of the patient and immediately transmit a warning message to a screen 28 of the control device 4. Subsequently the second network interface 14 can receive the medical device identification of the HSM 10 and forward it to the control device 4. The control device 4 searches through the aforementioned stored safety-relevant information for corresponding information about the HSM 10. If the control device 4 has found corresponding information, it controls a reprogramming of the HSM 10 in order to put it into a safe state during diagnosis with the MRT 6. The reprogramming can be undertaken for example on the basis of an instruction plan which the medical personnel can follow. The advantage of this is that the medical personnel can still undertake the programming manually but no specialized knowledge is needed for programming After the reprogramming of the HSM 10 the control device 4 restricts the possible diagnosis sequences with the MRT 6 and all operating practices of the MRT 6 to values of safety concern. Subsequently the control device guides the medical personnel through the diagnosis process with the MRT 6. After conclusion of the diagnosis process the control device can assist the medical personnel in returning the HSM 10 to its original programming in precisely the same manner as it did for programming

The load on the medical personnel is relieved by the method given above, since manual reprogramming is no longer necessary. Furthermore the medical personnel also no longer need to verify the programmed data.

Finally the control device 4 can also manage a number of implanted medical devices at the same time, so that as well as the HSM 10, other devices can also be implanted in the patient.

We claim:

1. A method for safeguarding an implanted medical device against electromagnetic radiation from a diagnostic device, comprising:
detecting the medical device by bidirectional communication with the diagnostic device;
determining a device type of the medical device with the diagnostic device;
checking whether the device type of the medical device may be operated without errors under the electromagnetic radiation of the diagnostic device; and
initiating a protective measure when the device type of the medical device cannot be operated without errors under the electromagnetic radiation.

2. The method as claimed in claim 1, wherein the protective measure comprises a reconfiguration of the medical device such that the medical device is operated without errors under the electromagnetic radiation of the diagnostic device.

3. The method as claimed in claim 2, further comprising putting the diagnostic device into operation after the reconfiguration of the medical device and subsequently returning the medical device to its original program.

4. The method as claimed in claim 1, wherein the device type of the medical device is able to be determined on the basis of an identifier stored in the medical device.

5. The method as claimed in claim 4, further comprising interrogating the identifier from the medical device and undertaking the determination of the device type of medical device based on the interrogated identifier.

6. The method as claimed in claim 1, wherein an entry is stored in a database indicating whether the device type of the medical device is operated without errors under the electromagnetic radiation.

7. The method as claimed in claim 6, further comprising retrieving the entry from the database on the diagnostic device and checking, on the basis of the retrieved entry, whether the device type of the medical device may be operated without errors under the electromagnetic radiation of the diagnostic device.

8. The method as claimed in claim 7, wherein a plurality of device type entries are retrieved from the database before the medical device is detected with the diagnostic device.

9. The method as claimed in claim 6, wherein the entry further specifies whether the device type of the medical device may be operated without errors under a specific configuration of the diagnostic device and/or under a specific configuration of the medical device.

10. The method as claimed in claim 1, wherein the protective measure comprises outputting a warning as to whether the device type of the medical device may be operated without errors under the electromagnetic radiation.

11. A control device for safeguarding an implanted medical device against electromagnetic radiation of a diagnostic device, comprising:
a network interface for bidirectional exchange of data with the medical device and is set up for carrying out a method as claimed in claim 1

12. The control device as claimed in claim 11, further comprising a network interface for receiving the entries of a database from a memory in which the database is stored.

13. An apparatus for diagnostic examination of a patient, comprising:
a control device as claimed in claim 11; and
a diagnostic device.
